(19) United States
(12) Patent Application Publication
(10) Pub. No.: US 2003/0191187 A1
(43) Pub. Date: Oct. 9, 2003

(54) Injectable pharmaceutical composition containing a non-steroidal anti-inflammatory drug and method for preparing the same

(76) Inventors: Fang Yu Lee, Taichung (TW); Shan Chiung Chen, Taichung (TW); Bin Ken Chen, Taichung (TW); Chiung Ju Tsai, Taichung (TW)

Correspondence Address: VENABLE, BAETJER, HOWARD AND CIVILETTI, LLP, P.O. BOX 34385, WASHINGTON, DC, 20043-9998, US

(21) Appl. No.: 10/109,859

(22) Filed: Apr. 1, 2002

(51) Int. Cl. A61K 31/192

(52) U.S. Cl. 514/570

(57) ABSTRACT

The present invention provides a stable pharmaceutical composition which contains an acetic acid class of non-steroidal anti-inflammatory drug (NSAID), a phosphate solution, an isotonic agent, and water. The pharmaceutical composition is particularly suitable for parenteral injection such as intravenous or intramuscular injection. The preferred NSAID is ketorolac tromethamine, which includes any racemic mixture of [−]S and [+]R ketorolac tromethamine. The preferred phosphate solution contains sodium phosphate monobasic (NaH₂PO₄) or potassium phosphate monobasic (KH₂PO₄), with or without crystalline water. The preferred isotonic agent is NaCl. The pharmaceutical composition is preferably at pH 6.0 to 8.5 and with osmolarity within 0.5 to 3.

### FIELD OF THE INVENTION

The present invention relates to an injectable pharmaceutical composition which contains an acetic acid class of non-steroidal anti-inflammatory drug (NSAID), a phosphate solution, an isotonic agent, and water; in particular, ketorolac tromethamine is the preferred NSAID, sodium phosphate monobasic (NaH₂PO₄) or potassium phosphate monobasic (KH₂PO₄), anhydrous or with hydrates, is the preferred phosphate for the phosphate solution, and NaCl is the preferred isotonic agent. The pharmaceutical composition is preferably at pH 6.0 to 8.5 and with osmolarity within 0.5 to 3 Osm. The present invention also relates to a method for preparing the injectable pharmaceutical composition. The injectable pharmaceutical composition of the present invention is stable and suitable for parenteral injection.

### BACKGROUND OF THE INVENTION

Non-steroidal anti-inflammatory drugs (NSAIDs) are a family of drugs that generally have analgesic, antipyretic, and anti-inflammatory activities. These activities derive from a common mechanism: the inhibition of cyclooxygenase, which is the critical enzyme for biosynthesis of prostaglandins, prostacyclin, and thromboxanes. Because prostaglandins are released in response to inflammatory stimuli, which in turn result in inflammatory responses (e.g., redness, pain, heat and swelling of tissue), inhibition of prostaglandins by NSAIDs results in analgesia. In the central nervous system, NSAIDs are antihyperalgesic through a direct action on the spinal cord.

Two NSAIDs, ketorolac and diclofenac, both belong to the acetic acid class of NSAIDs, are comparable to opioids in terms of providing pain relief. For example, the overall analgesic effect of 30 mg of ketorolac is equivalent to that of 6 to 12 mg of Morphine.

Ketorolac is a derivative of pyrrolizine carboxylic acid and is structurally related to tolmetin and zomepirac. The most commonly used form of ketorolac is ketorolac tromethamine, which is much more water soluble than the free acid form of ketorolac. The chemical name for ketorolac is (±)-5-benzoyl-2,3-dihydro-1H-pyrrolizine-1-carboxylic acid. When ketorolac is compounded with tromethamine (2-amino-2-(hydroxymethyl)-1,3-propanediol), its chemical structure is as follows:

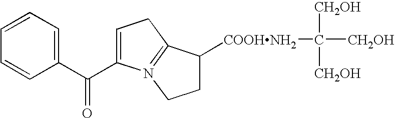

Ketorolac tromethamine has a pKa of 3.5 and an n-octanol/water partition coefficient of 0.26. The molecular weight of ketorolac tromethamine is 376.41. The white to off-white crystalline substance of ketorolac tromethamine discolors under prolonged exposure to light.

There are various dosage forms/formulations for ketorolac tromethamine. For example, U.S. Pat. No. 6,090,368 discloses a pharmaceutical composition comprising ketorolac tromethamine admixed with an aqueous bioadhesive cellulosic polymer containing microcrystalline particles. The pharmaceutical composition is particularly useful for use in nasal spray. U.S. Pat. No. 5,414,011 discloses an ophthalmic formulations consisting of ketorolac alone or in combination with an antibiotic drug, and a preservative system having a quaternary ammonium preservative and a nonionic polyoxyethylated octylphenol surfactant. U.S. Pat. No. 5,883,115 discloses a transdernal delivery of an eutomer of ketorolac. Ketorolac is a chiral drug which contains racemic mixture of [−]S form and [+]R form. The biological activity of ketorolac is with the S form. An eutomer is the stereoisomer of a chiral drug that exhibits greater pharmaceutical activity than its counterpart stereoisomer. In this case, the eutomer is the S form of ketorolac. U.S. Pat. No. 6,333,044 discloses a therapeutic composition of the racemic active form of ketorolac (i e., the S form), in combination with a pharmaceutically acceptable excipient or diluent, for use in intranasal administration.

The parenteral solutions of ketorolac tromethamine currently sold in the market contain sodium chloride (NaCl) and 10% (w/v) alcohol in the sterile solution. The parenteral solutions are clear and slightly yellow in color. Because of the 10% (w/v) alcohol content, the ketorolac tromethamine parenteral solution is contraindicated for intrathecal and epidural injections.

Also, ketorolac tromethamine parenteral solution is sensitive to light, which is due partially to the quality of alcohol used in the solution, i.e., the better the quality or the grade of alcohol, the less the sensitivity to light of the solution. However, without the alcohol, the ketorolac tromethamine parenteral solution is less stable, particularly for prolonged storage.

In the invention to be presented in the following sections, an injectable pharmaceutical composition is described. This injectable pharmaceutical composition is particularly design for intravenous or intramuscular injection of an acetic acid class of NSAID to patients for pain relief. The injectable pharmaceutical composition differs from the commercially available ketorolac tromethamine parenteral solution for it does not need to contain alcohol for stability and effectiveness. The injectable pharmaceutical contains a phosphate solution at 0.1 to 15 milliequivalent (meq), at pH 6.0-8.5, and at osmotic pressure of 0.3-5 Osm, which are also distinctively different from the commercially available ketorolac tromethamine parenteral solutions.

### SUMMARY OF THE INVENTION

The present invention provides an injectable pharmaceutical composition which contains an effective amount of a non-steroidal anti-inflammatory drug (NSAID); a phosphate solution; and water. The NSAID is an acetic acid class of NSAID, which include, but are not limited to, ketorolac, diclofenac, indomethacin, sulindac, etodolac, zomepirac, and tolmetin. Among this group of NSAIDs, ketorolac and/or a pharmaceutically acceptable salt of ketorolac, such as ketrolac tromethamine, is the preferred one. The NSAID constitutes about 0.1 to 15% by weight of the total injectable pharmaceutical composition.

The injectable pharmaceutical composition is preferred to be at pH of about 6.0 to 8.5, and most favorably, at 6.9 and 7.9. The osmolarity of the injectable pharmaceutical composition should be about 0.5 to 3 Osm. The injectable pharmaceutical composition is administered by parenteral injection, particularly intravenous and intramuscular injections.

The phosphate solution of the injectable pharmaceutical contains phosphate or salt of phosphate, or a combination thereof at any ratio. The phosphate or salt of phosphate can be anhydrous or with hydrates. The salt of phosphate includes, but is not limited to, the anhydrous or hydrous form of sodium phosphate dibasic, sodium phosphate monobasic, sodium phosphate tribasic, potassium phosphate monobasic, potassium phosphate dibasic, and potassium phosphate tribasic. The preferred salt of phosphate is the anhydrous or hydrous form of sodium phosphate monobasic or potassium phosphate monobasic. The phosphate solution is preferably at about 0.1-15 meg of said phosphate solution.

The pH of the injectable pharmaceutical composition is adjusted by a pH-adjusting agent which includes, but is not limited to, sodium hydroxide, potassium hydroxide, tromethamine, monoethanolamine, potassium citrate, triethanolamine, sodium citrate, diethanolamine, sodium bicarbonate, hydrochloride acid, tartaric acid, citric acid, lactic acid, and sodium lactate. The preferred pH-adjusting agent is NaOH and/or HCl.

The osmolarity of the injectable pharmaceutical composition is adjusted by an isotonic agent which is sodium chloride or potassium chloride, or a combination of both.

The injectable pharmaceutical composition does not contain alcohol. Optionally, an alcohol or isopropyl alcohol can be added to the injectable pharmaceutical composition. If isopropyl alcohol is added to the injectable pharmaceutical composition, the amount of isopropyl alcohol can not exceed 2% by volume.

The present invention also provides a method for preparing the injectable pharmaceutical composition which includes mixing the acetic acid class of NSAID, the phosphate solution, the isotonic agent, and the water to form the injectable pharmaceutical composition.

Additionally, the present invention provides a method for treating patients with pain which includes intravenously or intramuscularly injecting an effective amount of the injectable pharmaceutical composition described above to the patients. Similarly, the present invention includes an analgesic which contains an effective amount of the injectable pharmaceutical composition as shown above.

Finally, the present invention includes a stable pharmaceutical composition which contains (1) about 0.1 to 15% by weight of an acetic acid class of a non-steroidal anti-inflammatory drug (NSAID); (2) about 0.01 to 10% by weight of a phosphate solution; (3) about 0.1 to 10% by weight of an isotonic agent; (4) a sufficient amount of a pH-adjusting agent to adjust pH of said stable pharmaceutical composition to about 6.0-8.5, most favorably 6.9 to 7.9; and (5) about 0.01 to 100% by volume of water.

The preferred acetic acid class of NSAID is ketorolac, particularly ketorolac tromethamine. The preferred phosphate solution contains anhydrous or hydrous form of sodium phosphate monobasic (NaH₂PO₄) or potassium phosphate monobasic (KH₂PO₄). The preferred isotonic agent is NaCl. The preferred pH-adjusting agent is NaOH or HCl.

The stable pharmaceutical composition does not contain alcohol. Optionally, an alcohol or isopropyl alcohol can be added to the injectable pharmaceutical composition. If isopropyl alcohol is added to the injectable pharmaceutical composition, the amount of isopropyl alcohol can not exceed 2% by volume.

The stable pharmaceutical composition is prepared by mixing about 0.1 to 15% by weight of the NSAID; about 0.01 to 10% by weight of the phosphate solution; about 0.1 to 10% by weight of the isotonic agent; a sufficient amount of a pH-adjusting agent to adjust pH of said stable pharmaceutical composition to about 6.0-8.5, most favorably 6.9 to 7.9; and about 0.01 to 100% by volume of water.

The stable pharmaceutical composition can be used to treat patients with pain and as an analgesic.

### DETAILED DESCRIPTION OF THE INVENTION

The non-steroidal anti-inflammatory drugs (NSAIDs) have analgesic, antipyretic and anti-inflammatory activities. NSAIDs are widely used for treatment of minor discomfort and illness and many disease conditions such as cold, aches and pains, mild fever, osteoarthritis, rheumatoid arthritis, acute or severe pain, etc. NSAIDs can be categorized into acetylsalicylic acid, propionic acid, acetic acid, fenamate (anthranillic acid), nonacidic, and oxicam groups. Example of the acetylsalicyclic acid class of NSAIDs includes, but is not limited to, aspirin. Examples of the propionic acid class of NSAIDs include, but are not limited to, ibuprofen, ketoprofen, naproxen, oxaprozin. Examples of the acetic acid class of NSAIDs include, but are not limited to, ketorolac, diclofenac, indomethacin, sulindac, etodolac, and tolmetin. Examples of the fenamate class of NSAIDs include, but are not limited to, meclofenamate and mefenamic acid. Example of the non-acidic class of NSAIDs includes, but is not limited to, nabumetone. Examples of the oxicam class of NSAIDs include, but are not limited to piroxicam and meloxicam (oxicam). The drugs illustrated in each class of the NSAIDs share similar, although not identical, pharmacokinetic and pharmcodynamic characteristics.

One agent in the acetic acid group, ketorolac, has a potent analgesic activity at the opioid level and is indicated for management of moderately severe acute pain. Though in the management of severe pains, opioids are very potent pain relievers, they have the history of developing tolerance, drug abuse, physical and mental dependency, withdrawal symptoms and adverse effects, which make their uses controversial and highly regulated.

Contrary to opioids, ketorolac is a relatively safe and effective drug for use in pain relieves. Ketorolac is currently commercially available in oral tablets, and intravenous and intramuscular injection solutions for quick onset of acute pain relief. The serum concentration of ketorolac reaches a peak at about 2.9±1.8 minutes. A single dose of intramuscular injection of 60 mg of ketorolac reaches a peak in serum concentration about 30 to 60 minutes.

Ketorolac is a chiral drug which can be separated into two racemic structures, i.e., [−]S and [+]R ketorolac forms. The biological activity of ketorolac is associated with the S-form. The term “ketorolac” as used herein refers to S-form, R-form, or a racemic mixture of ketorolac. The racemic mixture of the [−]S and [+]R isomers is currently used in the marketed oral, ophthalmic, intravenous and intramuscular pharmaceutical products.

The ketorolac free acid has low water solubility. However, one salt form of ketorolac, i.e., ketorolac tromethamine, has demonstrated enhanced solubility in water. Ketorolac tromethamine may exist in three crystal forms. All forms are equally soluble in water. Ketorolac tromethamine dissociates at the physiologic pH to anionic ketorolac. Pharmacokinetic behavior of ketorolac can be described using either the two- or three-compartmental models. Once in the circulation, ketorolac tromethamine is 99% plasma protein bound with a terminal elimination half-life of 3.8-6.3 hours in young adults and 4.7-8.6 hours in geriatric patients. Ketorolac tromethamine is largely metabolized in the liver to hydroxylated and conjugated metabolites. The metabolites and some unchanged drug are excreted in the urine. The onset of the anti-inflammatory, analgesic, and antipyretic effects starts within 30-60 minutes after the intramuscular administration. The maximum effect is reached 1-2 hours after the intravenous or intramuscular administrations. The therapeutic effects of ketorolac tromethamine last for 4-6 hours.

The currently commercially available ketorolac tromethamine perenteral solutions contain about 10% alcohol. However, the use of alcohol in the perenteral solutions creates certain problems which can limit the usage of ketorolac tromethamine for injection. For example, ketorolac tromethamine is sensitive to light and the sensitivity of ketorolac tromethamine is worsen if the alcohol used in the solution is not in top quality. On the other hand, if a top grade alcohol is used in the injection solution, the cost for making the ketorolac tromethamine injection solution increases which affects the competitiveness of the products in the market. In addition, high content of alcohol causes irritation of skin at the injection site and delay in drug absorption. It also induces drug-drug interactions when ketorolac tromethamine is administered together with other pharmaceutical products. High content of alcohol in the solution also causes precipitation of the ketorolac tromethamine in the solution which affects the stability and effectiveness of the products.

The use of isopropyl alcohol in the ketorolac tromethamine injection solution also creates problems. In particular, isopropyl alcohol increases the osmotic pressure of the injection solution, which, in turn causes pain or irritation to the patients at the site of the injection. If the concentration of isopropyl alcohol is too high, it may cause hemolysis in patients.

In the present invention, a stable and injectable pharmaceutical composition which contains an acetic acid class of NSAID is described. The pH value of the injectable pharmaceutical composition is maintained at 6.0-8.5, preferably 6.9-7.9. When the pH values of the ketorolac tromethamine solutions are lower than 6, the NSAID precipitates from the solutions. When the pH is higher than 8.5, the color of the solutions changes.

The osmolarity of the injectable pharmaceutical composition is maintained at 0.5-3 Osm. Higher osmotic pressure produces pain and increases skin irritation at the site of injection. It may also affect the rate of drug absorption. If the osmotic pressure is too high, it may cause hemolysis in intravenous injection. The term “osmolarity,” and “osmotic pressure” are used interchangeably in this application. Also, the term “isotonic” as used herein is referred to as pertaining to a solution characterized by having equal osmotic pressure as that in the mammalian blood.

Other than the NSAID, the injectable pharmaceutical composition of the present invention contains the following components:

(1). A Phosphate Solution:

The phosphate solution contains anhydrous or hydrous phosphate and/or salt of phosphate. The phosphate solution is preferably maintained at 0.1-10 milliequivalent (“meq”). The anhydrous or hydrous form of phosphate and the salt of phosphate include, but are not limited to, phosphoric acid (H₃PO₄), sodium phosphate tribasic (Na₃PO₄), sodium phosphate monobasic (NaH₂PO₄), sodium phosphate dibasic (Na₂HPO₄), potassium phosphate tribasic (K₃PO₄), potassium phosphate monobasic (KH₂PO₄), potassium phosphate dibasic (K₂HPO₄).

(2). pH-adjusting Agent:

To maintain the injectable pharmaceutical composition at pH 6.0 to 8.5, the following pH-Adjusting agent are employed:

(A). To increase the pH value of the injectable pharmaceutical composition, an alkaline agent is used, which includes, but is not limited to, sodium hydroxide (NaOH), potassium hydroxide (KOH), tromethamine, monoethanolamine, diethanolamine, sodium bicarbonate (NaH₂CO₃) and other organic bases.

(B). To decrease the pH value of the injectable pharmaceutical composition, an acidic agent is used, which includes, but is not limited to, hydrochloric acid (HCl), citric acid, tartaric acid, lactic acid and other organic acids.

The pH-adjusting agents can be used individually or in combination. The total concentrations of the pH adjusting agents are within the range of 0.001-5% by weight.

(3). An Isotonic Agent:

The injectable pharmaceutical composition is adjusted to within 0.5 to 3.0 Osmolarity, which is equivalent or similar to the osmotic pressure in the mammalian blood by an isotonic agent. The isotonic agent includes, but is not limit to, sodium chloride, potassium chloride, and/or other conventionally known isotonic agents.

(4). Water:

The injectable pharmaceutical composition is preferably dissolved in water, particularly sterile water. Optionally, ethanol or isopropyl alcohol can be added to the water. The addition of ethanol or isopropyl alcohol is not an absolute requirement for producing the stable and injectable pharmaceutical composition as described in the present invention. When isopropyl alcohol is used, it is preferred that the concentration of isopropyl alcohol does not exceed 2%. If the isopropyl alcohol concentration exceeds 35%, it may induce hemolysis in patients.

The injectable pharmaceutical composition is prepared by the following procedures, which are in compliance with the Food and Drug Administration of the United States Class 1 Good Manufacturing Practice (cGMP):

(1). Add an appropriate amount of the phosphate or the salt of phosphate, anhydrous or with hydrate, as described in the above “phosphate solution” section, to an appropriate amount of water, stir until the phosphate is dissolved.

(2). Add an appropriate amount of an acetic acid class of NSAID to the solution in (1); stir until the NSAID is dissolved.

(3). Optionally, add an appropriate amount of an isotonic agent, as described in the above “isotonic agent” section, to the solution in (2); stir until the isotonic agent is dissolve.

(4). Measure the pH of the solution in (3). If the pH is below 6.0, add an appropriate amount of the alkaline agent as described in (A) of the above “pH-adjusting agent” to the solution. If the pH is above 8.5, add an appropriate amount of the acidic agent as described in (B) of the above “pH-adjusting agent” to the solution. Cloudiness is observed when the pH of the solution is below 6. The solution will become clear after the pH adjustment to between 6.0-8.5.

(5). Sterilize the solution of (4) by passing the solution through a 0.22 μm filter.

(6). Dispense the desired quantity of the solution mentioned above into a sterilized container; sterilize the solution in the sterilized containers by autoclaving at high pressure at 121° C. for 20 minutes.

(7). If the solution is in an ampoule, conduct a methylene blue test for quality control in terms of leakage. This step can be omitted for products in vials.

(8). Wipe to clean the containers. Inspect the clarity of the solution to determine whether particulate and/or foreign matters, such as cotton fibers and crystals, are in the vial or ampoule by using an automatic light projection detector for ampoules.

(9). Label, package and store the finished products.

The following equipment are employed to determine the quality of the final products: (1) High performance liquid chromatography (HPLC) is used to determine the amount of NSAID in the injectable pharmaceutical composition; (2) pH meter is used to determine the pH value of the injectable pharmaceutical composition; (3) Osmometer is used to determine the osmotic pressure of the injectable pharmaceutical composition; (4) Atomic Absorption spectrometer is used to determine the Na⁺or K⁺concentration; and (5) Automatic Light Projection Detector by EISAI Co., Ltd., is used to detect impurity such as cotton fibers and crystals in the vial or ampoule

The manufacturing process is further depicted in the following flow chart (Table 1):

TABLE 1
Flow Chart of Manufacturing Process

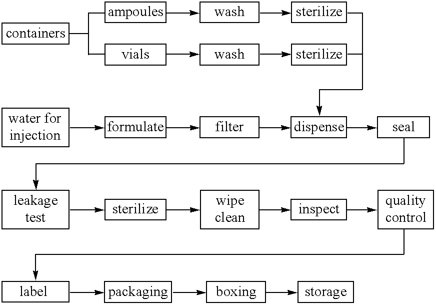

The following example is illustrative, but not limiting the scope of the present invention. Reasonable variations, such as those occur to reasonable artisan, can be made herein without departing from the scope of the present invention.

### EXAMPLE 1

### Preparation of the Pharmaceutical Composition of the Present Invention

The pharmaceutical composition of the present invention was prepared as follows:

| | | |
|---|---|---|
| Ketorolac tromethamine | 1800 | g |
| KH₂PO₄ | 300 | g |
| NaCl | 261 | g |

| | |
|---|---|
| NaOH or HCl | Adequate amount |

| | | |
|---|---|---|
| Water for Injection | qs to 60 | L |

Procedures:

(1) Add KH₂PO₄ to 50 L of water; stir until KH₂PO₄ is dissolved.

(2) Add ketorolac tromethamine to the solution of (1); stir until ketorolac tromethamine is dissolved.

(3) Add NaCl to the solution of (2); stir until the NaCl is dissolved.

(4) Adjust the pH of the solution of (3) to 6.9˜7.9 using an adequate amount of NaOH or HCl.

(5) Add the volume of the solution of (4) up to 60 L using an adequate amount of water.

(6) Filter the solution of (5) through a 0.22 μm filter.

(7) Dispense the filtered solution into a sterile container, such as an ampoule or vial; seal the container.

(8) Sterilize the solution in the container by autoclaving at 121° C. for 20 minutes.

(9) After the container is cool down, wipe clean the container; inspect the container for any particulate or foreign matters; release the product.

### EXAMPLE 2

### Preparation of the Pharmaceutical Composition of the Present Invention

The pharmaceutical composition of the present invention was prepared as follows:

| | | |
|---|---|---|
| Ketorolac tromethamine | 1800 | g |
| KH₂PO₄ | 300 | g |
| NaCl | 261 | g |

| | |
|---|---|
| Tromethamine or HCl | Adequate amount |

| | | |
|---|---|---|
| Water for Injection | Qs to 60 | L |

Procedures:

(1) Add KH₂PO₄ to 50 L of Water for Injection and; stir until KH₂PO₄ is dissolved.

(2) Add ketorolac tromethamine to the solution of (1); stir until ketorolac tromethamine is dissolved.

(3) Add NaCl to the solution of (2); stir until the NaCl is dissolved.

(4) Adjust the pH of the solution of (3) to 6.9˜7.9 using an adequate amount of tromethamine or HCl.

(5) Add the volume of the solution of (4) up to 60 L using an adequate amount of water.

(6) Filter the solution of (5) through a 0.22 μm filter.

(7) Dispense the filtered solution into a sterile container, such as an ampoule or vial; seal the container.

(8) Sterilize the containers by autoclaving at 121° C. for 20 minutes.

(9) After the container is cool down, wipe clean the container; inspect the container for any particulate or foreign matters; release the product.

### EXAMPLE 3

### Preparation of the Pharmaceutical Composition of the Present Invention

The pharmaceutical composition of the present invention was prepared as follows:

| | | |
|---|---|---|
| Ketorolac tromethamine | 1800 | g |
| Na₂HPO₄ ·12H₂O | 360 | g |
| NaCl | 261 | g |

| | |
|---|---|
| NaOH or HCl | adequate amount |

| | | |
|---|---|---|
| Water for Injection | qs to 60 | L |

Procedures:

(1) Add Na₂HPO₄.12H_{O} to 50 L of water; stir until Na₂HPO₄.12H₂O is dissolved.

(2) Add ketorolac tromethamine to the solution of (1); stir until ketorolac tromethamine is dissolved.

(3) Add NaCl to the solution of (2); stir until the NaCl is dissolved.

(4) Adjust the pH of the solution of (3) to 6.9˜7.9 using an adequate amount of NaOH or HCl.

(5) Add the volume of the solution of (4) up to 60 L using an adequate amount of water.

(6) Filter the solution of (5) through a 0.22 μm filter.

(7) Dispense the filtered solution into a sterile container, such as an ampoule or vial; seal the container.

(8) Sterilize the containers by autoclaving at 121° C. for 20 minutes.

(9) After the container is cool down, wipe clean the container; inspect the container for any particulate or foreign matters; release the product. EXAMPLE 4

### Preparation of the Pharmaceutical Composition of the Present Invention

The pharmaceutical composition of the present invention was prepared as follows:

| | | |
|---|---|---|
| ketorolac tromethamine | 1800 | g |
| Na₃PO₄ | 240 | g |
| NaCl | 261 | g |

| | |
|---|---|
| NaOH or HCl | adequate amount |

| | | |
|---|---|---|
| Water for Injection | qs to 60 | L |

Procedures:

(1) Add Na₃PO₄ to 50 L of water; stir until Na₃PO₄ is dissolved.

(2) Add ketorolac tromethamine to the solution of (1); stir until ketorolac tromethamine is dissolved.

(3) Add NaCl to the solution of (2); stir until the NaCl is dissolved.

(4) Adjust the pH of the solution of (3) to 6.9˜7.9 using an adequate amount of NaOH or HCl.

(5) Add the volume of the solution of (4) up to 60 L using an adequate amount of water.

(6) Filter the solution of (5) through a 0.22 μm filter.

(7) Dispense the filtered solution into a sterile container, such as an ampoule or vial; seal the container.

(8) Sterilize the containers by autoclaving at 121° C. for 20 minutes.

(9) After the container is cool down, wipe clean the container; inspect the container for any particulate or foreign matters; release the product.

### EXAMPLE 5

### Preparation of the Pharmaceutical Composition of the Present Invention

The pharmaceutical composition of the present invention was prepared as follows:

| | | |
|---|---|---|
| Ketorolac tromethamine | 1800 | g |
| KH₂PO₄ | 300 | g |
| NaCl | 261 | g |

| | |
|---|---|
| KOH or HCl | adequate amount |

| | | |
|---|---|---|
| Water for Injection | qs to 60 | L |

Procedures:

(1) Add KH₂PO₄ to 50 L of water; stir until KH₂PO₄ is dissolved.

(2) Add ketorolac tromethamine to the solution of (1); stir until ketorolac tromethamine is dissolved.

(3) Add NaCl to the solution of (2); stir until the NaCl is dissolved.

(4) Adjust the pH of the solution of (3) to 6.9˜7.9 using an adequate amount of KOH or HCl.

(5) Add the volume of the solution of (4) up to 60 L using an adequate amount of water.

(6) Filter the solution of (5) through a 0.22 μm filter.

(7) Dispense the filtered solution into a sterile container, such as an ampoule or vial; seal the container.

(8) Sterilize the containers by autoclaving at 121° C. for 20 minutes.

(9) After the container is cool down, wipe clean the container; inspect the container for any particulate or foreign matters; release the product

### EXAMPLE 6

### Preparation of the Pharmaceutical Composition of the Present Invention

The pharmaceutical composition of the present invention was prepared as follows:

| | | |
|---|---|---|
| Ketorolac tromethamine | 1800 | g |
| NaH₂PO₄ · 2H₂O | 342 | g |
| NaCl | 261 | g |

| | |
|---|---|
| NaOH or HCl | adequate amount |

| | | |
|---|---|---|
| Water for Injection | qs to 60 | L |

Procedures:

(1) Add NaH₂PO₄.2H₂O to 50 L of water; stir until NaH₂PO₄ .2H₂O is dissolved.

(2) Add ketorolac tromethamine to the solution of (1); stir until ketorolac tromethamine is dissolved.

(3) Add NaCl to the solution of (2); stir until the NaCl is dissolved.

(4) Adjust the pH of the solution of (3) to 6.9˜7.9 using an adequate amount of NaOH or HCl.

(5) Add the volume of the solution of (4) up to 60 L using an adequate amount of water.

(6) Filter the solution of (5) through a 0.22 μm filter.

(7) Dispense the filtered solution into a sterile container, such as an ampoule or vial; seal the container.

(8) Sterilize the containers by autoclaving at 121° C. for 20 minutes.

(9) After the container is cool down, wipe clean the container; inspect the container for any particulate or foreign matters; release the product.

### EXAMPLE 7

### Preparation of the Pharmaceutical Composition of the Present Invention

The pharmaceutical composition of the present invention was prepared as follows:

| | | |
|---|---|---|
| ketorolac tromethamine | 1800 | g |
| K₂HPO₄ | 300 | g |
| NaCl | 261 | g |

| | |
|---|---|
| NaOH or citric acid | adequate amount |

| | | |
|---|---|---|
| Water for Injection | qs to 60 | L |

Procedures:

(1) Add K₂HPO₄ to 50 L of water; stir until K₂HPO₄ is dissolved.

(2) Add ketorolac tromethamine to the solution of (1); stir until ketorolac tromethamine is dissolved.

(3) Add NaCl to the solution of (2); stir until the NaCl is dissolved.

(4) Adjust the pH of the solution of (3) to 6.9˜7.9 using an adequate amount of NaOH or citric acid.

(5) Add the volume of the solution of (4) up to 60 L using an adequate amount of water.

(6) Filter the solution of (5) through a 0.22 μm filter.

(7) Dispense the filtered solution into a sterile container, such as an ampoule or vial; seal the container.

(8) Sterilize the containers by autoclaving at 121° C. for 20 minutes.

(9) After the container is cool down, wipe clean the container; inspect the container for any particulate or foreign matters; release the product

### EXAMPLE 8

### Preparation of the Pharmaceutical Composition of the Present Invention

The pharmaceutical composition of the present invention was prepared as follows:

| | | |
|---|---|---|
| ketorolac tromethamine | 1800 | g |
| NaH₂PO₄ · 2H₂O | 342 | g |
| KH₂PO₄ | 300 | g |
| NaCl | 522 | g |

| | |
|---|---|
| NaOH or citric acid | adequate amount |

| | | |
|---|---|---|
| Water for Injection | qs to 60 | L |

Procedures:

(1) Add NaH₂PO₄.2H₂O and KH₂PO₄ to 50 L of water; stir until NaH₂PO₄.2H₂O and KH₂PO₄ are dissolved.

(2) Add ketorolac tromethamine to the solution of (1); stir until ketorolac tromethamine is dissolved.

(3) Add NaCl to the solution of (2); stir until the NaCl is dissolved.

(4) Adjust the pH of the solution of (3) to 6.9˜7.9 using an adequate amount of NaOH or citric acid.

(5) Add the volume of the solution of (4) up to 60 L using an adequate amount of water.

(6) Filter the solution of (5) through a 0.22 μm filter.

(7) Dispense the filtered solution into a sterile container, such as an ampoule or vial; seal the container.

(8) Sterilize the containers by autoclaving at 121° C. for 20 minutes.

(9) After the container is cool down, wipe clean the container; inspect the container for any particulate or foreign matters; release the product

### RESULTS OF EXAMPLES 1-8

The formulations of EXAMPLES 1-8 are summarized in the following Table 2:

TABLE 2
Formulations of Examples 1-8

| | EXAMPLE | | | | | | | |
|---|---|---|---|---|---|---|---|---|
| Component | 1 | 2 | 3 | 4 | 5 | 6 | 7 | 8 |
| Ketorolac | 1800 g | 1800 g | 1800 g | 1800 g | 1800 g | 1800 g | 1800 g | 1800 g |
| tromethamine | | | | | | | | |
| NaCl | 261 g | 261 g | 261 g | 261 g | 261 g | 261 g | 261 g | 522 g |
| KH₂PO₄ | 300 g | 300 g | — | — | 300 g | — | — | 300 g |
| NaOH or HCl | Adequate | — | adequate | adequate | — | Adequate | — | — |
| | amount | | amount | amount | | amount | | |
| tromethamine or | — | Adequate | — | — | — | — | — | — |
| HCl | | amount | | | | | | |
| Na₂HPO₄.12H₂O | — | — | 360 g | — | — | — | — | — |
| Na₃PO₄ | — | — | — | 240 g | — | — | — | — |
| KOH or HCl | — | — | — | — | Adequate | — | — | — |
| | | | | | amount | | | |
| NaH₂PO₄.2H₂O | — | — | — | — | — | 342 g | — | 342 g |
| K₂HPO₄ | — | — | — | — | — | — | 300 g | — |
| NaOH or citric acid | — | — | — | — | — | — | adequate | adequate |
| | | | | | | | amount | amount |
| Water for | qs to 60 | Qs to 60 | qs to 60 | qs to 60 | Qs to 60 | qs to 60 | qs to 60 | qs to 60 |
| Injection | L | L | L | L | L | L | L | L |

The following tests were carried out on the ketorolac tromethamine solutions prepared as described in the examples: ketorolac tromethamine assay (measured by high performance liquid chromatography (HPLC)), pH, relative osmotic pressure, sodium content (measured by atomic absorption and atomic emission spectroscopy), phosphate content, manufacturing reject rate, appearance, sterility and pyrogen. The results are summarized as follows:

TABLE 3
Test Results of Examples 1-8.

| | EXAMPLE | | | | | | | |
|---|---|---|---|---|---|---|---|---|
| Test | 1 | 2 | 3 | 4 | 5 | 6 | 7 | 8 |
| assay (%) | 99.7 | 99.1 | 98.6 | 99.4 | 99.5 | 98.7 | 98.1 | 97.9 |
| PH | 7.40 | 7.35 | 7.47 | 7.60 | 7.35 | 7.51 | 7.20 | 7.30 |
| relative osmotic | 0.97 | 0.90 | 0.80 | 1.02 | 0.86 | 1.10 | 1.01 | 1.05 |
| pressure | | | | | | | | |
| sodium content | 0.223 | 0.208 | 0.184 | 0.320 | 0.162 | 0.219 | 0.172 | 0.158 |
| (%) | | | | | | | | |
| Phosphate | 5.0 | 5.0 | 6.0 | 4.0 | 5.0 | 5.7 | 5.0 | 5.7 |
| content (mg) | | | | | | | | |
| Rejection rate | 0 | 0.5 | 3.2 | 3.4 | 3.5 | 1.2 | 1.3 | 0.5 |
| (%) | | | | | | | | |

| | |
|---|---|
| Appearance | Light yellow, clear solution |
| sterility test | Sterile |
| Pyrogen | Negative |

Conclusion:

As shown in Table 3, the combined use of different kinds and amounts of phosphate/salts of phosphate (anhydrous or with hydrates) in Examples 1-8 results in similarly safe, stable, and high quality of injectable pharmaceutical composition, as demonstrated by the clarity of the solutions (i.e., light yellow transparent solution); the low rejection rates (%), and the quality of the solution (i.e., by sterilization and negative pyrogen). Among the EXAMPLES, EXAMPLE 1 has the best attributes over the rest of the examples.

We claim:

1. An injectable pharmaceutical composition comprising
an effective amount of a non-steroidal anti-inflammatory drug (NSAID), wherein said NSAID is an acetic acid class of NSAID;
a phosphate solution;
an isotonic agent; and
water;
wherein said injectable pharmaceutical composition is at a pH of about 6.0 to 8.5 and an osmolarity of about 0.5 to 3 osm; and
wherein said injectable pharmaceutical composition is administered to patients by parenteral injection.

2. The injectable pharmaceutical composition according to claim 1, wherein said acetic acid class of NSAID is one selected from the group consisting of ketorolac, diclofenac, indomethacin, sulindac, etodolac, zomepirac, and tolmetin.

3. The injectable pharmaceutical composition according to claim 1, wherein said acetic acid class of NSAID comprises ketorolac, a pharmaceutically acceptable salt of ketorolac, or a combination thereof.

4. The injectable pharmaceutical composition according to claim 3, wherein said pharmaceutical acceptable salt of ketorolac is ketorolac tromethamine.

5. The injectable pharmaceutical composition according to claim 1, wherein said pharmaceutical composition comprises 0.1 to 15% by weight of said acetic acid class of NSAID.

6. The injectable pharmaceutical composition according to claim 1, wherein said phosphate solution comprises anhydrous or hydrous form of phosphate or salt of phosphate, or a combination thereof.

7. The injectable pharmaceutical composition according to claim 6, wherein said anhydrous or hydrous form of salt of phosphate is at least one selected from the group consisting of sodium phosphate dibasic, sodium phosphate monobasic, sodium phosphate tribasic, potassium phosphate monobasic, potassium phosphate dibasic, and potassium phosphate tribasic.

8. The injectable pharmaceutical composition according to claim 1, wherein said phosphate solution comprises anhydrous or hydrous form of sodium phosphate monobasic or potassium phosphate monobasic.

9. The injectable pharmaceutical composition according to claim 1, wherein said pharmaceutical composition comprises about 0.1-15 meg of said phosphate solution.

10. The injectable pharmaceutical composition according to claim 1, wherein said pH of said pharmaceutical composition is adjusted by a pH-adjusting agent which is at least one selected from the group consisting of sodium hydroxide, potassium hydroxide, tromethamine, monoethanolamine, potassium citrate, triethanolamine, sodium citrate, diethanolamine, sodium bicarbonate, hydrochloride acid, citric acid, tartaric acid, lactic acid, and sodium lactate.

11. The injectable pharmaceutical composition according to claim 1, wherein said pH is between 6.9 and 7.9.

12. The injectable pharmaceutical composition according to claim 9, wherein said pH-adjusting agent is NaOH or HCl.

13. The injectable pharmaceutical composition according to claim 1, wherein said an isotonic agent is at least one selected from the group consisting of sodium chloride and potassium chloride.

14. The injectable pharmaceutical composition according to claim 1, wherein said injectable pharmaceutical composition is administered intravenously or intramuscularly.

15. The injectable pharmaceutical composition according to claim 1 does not contain alcohol.

16. A method for preparing an injectable pharmaceutical composition comprising:
mixing said acetic acid class of NSAID; said phosphate solution; said isotonic agent, and said water according to claim 1 to form said injectable pharmaceutical composition.

17. The method according to claim 16, wherein said injectable pharmaceutical composition comprises about 0.1-15% by weight of said acetic acid class of NSAID.

18. The method according to claim 17, wherein said injectable pharmaceutical composition comprises about 0.1 meq to 15 meq of said phosphate solution.

19. A method for treating patients with pain comprising intravenously or intramuscularly injecting an effective amount of said injectable pharmaceutical composition according to claim 1 to said patients.

20. An analgesic comprising an effective amount of said injectable pharmaceutical composition according to claim 1.

21. A stable pharmaceutical composition comprising:
about 0.1 to 15% by weight of an acetic acid class of a non-steroidal anti-inflammatory drug (NSAID);
about 0.01 to 10% by weight of a phosphate solution;
about 0.1 to 10% by weight of an isotonic agent;
a sufficient amount of a pH-adjusting agent which adjusts pH of said stable pharmaceutical composition to about 6.0-8.5; and
about 0.01 to 100% by volume of water.

22. The stable pharmaceutical composition according to claim 21, wherein said acetic acid class of NSAID is ketorolac, a pharmaceutically acceptable salt of ketorolac, or a combination thereof.

23. The stable pharmaceutical composition according to claim 21, wherein said pharmaceutically acceptable salt of ketorolac is ketorolac tromethamine.

24. The stable pharmaceutical composition according to claim 21, wherein said phosphate solution comprises anhydrous or hydrous form of sodium phosphate monobasic (NaH₂PO₄) or potassium phosphate monobasic (KH₂PO₄).

25. The stable pharmaceutical composition according to claim 21, wherein said isotonic agent is NaCl.

26. The stable pharmaceutical composition according to claim 21, wherein said pH-adjusting agent is NaOH or HCl.

27. The stable pharmaceutical composition according to claim 21, wherein said pH is between 6.9 and 7.9.

28. The stable pharmaceutical composition according to claim 21 does not contain alcohol.

29. A method for preparing a stable pharmaceutical composition comprising:
mixing the NSAID; the phosphate solution; the isotonic agent; the pH-adjusting agent; and the water according to claim 21.

30. A method for treating patients with pain comprising:
intravenously or intramuscularly injecting an effective amount of said stable pharmaceutical composition according to claim 21 to said patients.

31. An analgesic comprising an effective amount of said stable pharmaceutical composition according to claim 21.
